(19) United States
(12) Patent Application Publication
(10) Pub. No.: US 2025/0072754 A1
(43) Pub. Date: Mar. 6, 2025

(54) MEDICAL DEVICES WITH AN OPTICAL MODULE

(71) Applicant: Cardiac Pacemakers, Inc., St Paul, MN (US)

(72) Inventors: Keith R. Maile, New Brighton, MN (US); Michael J. Kane, St Paul, MN (US); Nicholas Anderson, St Paul, MN (US)

(21) Appl. No.: 18/817,871

(22) Filed: Aug. 28, 2024

Related U.S. Application Data
(60) Provisional application No. 63/535,258, filed on Aug. 29, 2023.

(51) Int. Cl. A61B 5/00

(52) U.S. Cl. CPC A61B 5/0017; A61B 5/0031; A61B 5/686; A61B 5/7225; A61B 2562/185

(57) ABSTRACT

An implantable medical device (IMD) that includes a first light emitter arranged to generate a first beam of emitted light, a second light emitter arranged to generate a second beam of emitted light, a light detector arranged to sense backscattered light, and at least one optical layer comprising a beam steering feature and/or a cross-talk feature.

### CROSS REFERENCE TO RELATED APPLICATION

This application claims priority to U.S. patent application 63/535,258, filed on Aug. 29, 2023, which is herein incorporated by reference in the entirety.

### TECHNICAL FIELD

Instances of the present disclosure relate to medical devices and systems for sensing physiological parameters using an optical sensor.

### BACKGROUND

Implantable medical devices (IMDs) may be configured to sense physiological parameters and/or provide therapy. Examples of IMDs include implantable cardiac monitors, implantable loop recorders, and the like, which can be configured to be subcutaneously implanted in a patient for monitoring one or more physiological parameters such as physiological parameters associated with the heart and/or the lungs.

### SUMMARY

In Example 1, an implantable medical device (IMD) that includes a first light emitter arranged to generate a first beam of emitted light, a second light emitter arranged to generate a second beam of emitted light, a light detector arranged to sense backscattered light responsive to the first beam and the second beam, and at least one optical layer comprising a beam steering feature and/or a cross-talk feature.

In Example 2, the IMD of Example 1, wherein the beam steering feature is a lens or an optical grating.

In Example 3, the IMD of Example 1, wherein the beam steering feature is a Fresnel lens.

In Example 4, the IMD of any of Examples 1-3, further including a first wall positioned between the first light emitter and the light detector, wherein the first wall is arranged to at least partially block non-backscattered light.

In Example 5, the IMD of Example 4, wherein the first wall is positioned between the second light emitter and the detector.

In Example 6, the IMD of any of Examples 1-4, further including a second wall positioned between the second light emitter and the light detector.

In Example 7, the IMD of any of Examples 1-6, wherein the cross-talk feature is an area of dark material.

In Example 8, the IMD of any of Examples 1-7, wherein the first emitter and the second emitter are part of a single integrated circuit package.

In Example 9, the IMD of any of Examples 1-8, wherein the first emitter and the second emitter are light-emitting diodes.

In Example 10, the IMD of any of Examples 1-9, wherein the first emitter, the second emitter, and the detector have respective top surfaces that face a bottom surface of the optical layer.

In Example 11, the IMD of any of Examples 1-10, wherein the cross-talk feature includes multiple filter layers.

In Example 12, the IMD of any of Examples 1-11, wherein the first light emitter, the second light emitter, and the detector are coupled to a single circuit board.

In Example 13, the IMD of any of Examples 1-12, wherein an edge of the first light emitter is positioned 3-6 mm from an adjacent edge of the detector.

In Example 14, the IMD of any of Examples 1-13, wherein the first beam comprises infrared light, wherein the second beam comprises red light.

In Example 15, the IMD of any of Examples 1-14, wherein the emitter and the detector are part of an optical module positioned within a housing of the IMD.

In Example 16, an IMD includes an optical module. The optical module includes a first light emitter arranged to generate a first beam of emitted light, a second light emitter arranged to generate a second beam of emitted light, a light detector arranged to sense backscattered light responsive to the first beam and the second beam, and at least one optical layer comprising a beam steering feature and/or a cross-talk feature.

In Example 17, the IMD of Example 16, wherein the at least one optical layer includes the beam steering feature, wherein the beam steering feature is a lens or an optical grating.

In Example 18, the IMD of Example 16, wherein the at least one optical layer includes the beam steering feature, wherein the beam steering feature is a Fresnel lens.

In Example 19, the IMD of Example 16, further including a first wall positioned between the first light emitter and the light detector, wherein the first wall is arranged to at least partially block non-backscattered light.

In Example 20, the IMD of Example 19, wherein the first wall is positioned between the second light emitter and the detector.

In Example 21, the IMD of Example 19, further including a second wall positioned between the second light emitter and the light detector.

In Example 22, the IMD of Example 16, wherein the at least one optical layer includes the cross-talk feature, wherein the cross-talk feature is an area of dark material.

In Example 23, the IMD of Example 16, wherein the first emitter and the second emitter are light-emitting diodes.

In Example 24, the IMD of Example 16, wherein the at least one optical layer includes the cross-talk feature, wherein the cross-talk feature includes multiple filter layers.

In Example 25, the IMD of Example 16, wherein the first light emitter, the second light emitter, and the detector are coupled to a single circuit board.

In Example 26, the IMD of Example 16, wherein an edge of the first light emitter is positioned 3-6 mm from an adjacent edge of the detector.

In Example 27, the IMD of Example 16, wherein the first beam comprises infrared light, wherein the second beam comprises red light.

In Example 28, the IMD of Example 16, further including a housing and electrodes, wherein the optical module is positioned within the housing, wherein the electrodes are coupled to the housing.

In Example 29, the IMD of Example 16, wherein the at least one optical layer is part of a single window, wherein the single window is arranged such that the first beam, the second beam, and the backscattered light pass through the single window.

In Example 30, the IMD of Example 29, further including a single seal surrounding a perimeter of the single window.

In Example 31, the IMD of Example 16, wherein the optical module is configured to consume a maximum of 5 milliamps of power during operation.

In Example 32, the IMD of Example 16, wherein the light detector is a single light detector, wherein the light detector is the only light detector of the IMD.

In Example 33, an IMD with an optical module that includes: a first light emitter arranged to generate a first beam of emitted light, a second light emitter arranged to generate a second beam of emitted light, a light detector arranged to sense backscattered light responsive to the first beam and the second beam, and means for steering the first beam and the second beam.

In Example 34, the IMD of Example 33, further including means for reducing interference with the backscattered light.

In Example 35, the IMD of Example 34, wherein the first beam comprises infrared light, wherein the second beam comprises red light.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration depicting an illustrative medical system, in accordance with certain instances of the present disclosure.

FIG. 2 shows an implantable medical device, in accordance with certain instances of the present disclosure.

FIG. 3 shows a side, cut-away view of an optical sensor assembly, in accordance with certain instances of the present disclosure.

FIG. 4 shows a side, cut-away view of a portion of an optical sensor assembly, in accordance with certain instances of the present disclosure.

FIG. 5 shows a top view of a portion of an optical sensor assembly, in accordance with certain instances of the present disclosure.

FIG. 6A and FIG. 6B show different arrangements of an optical sensor assembly, in accordance with certain instances of the present disclosure.

While the invention is amenable to various modifications and alternative forms, specific embodiments have been shown by way of example in the drawings and are described in detail below. The intention, however, is not to limit the invention to the particular instances described. On the contrary, the invention is intended to cover all modifications, equivalents, and alternatives falling within the scope of the invention as defined by the appended claims.

### DETAILED DESCRIPTION

Optical sensor devices can be used to measure physiological parameters such as oxygen saturation in blood. For example, an optical sensor device can be placed on a person's body part (e.g., finger, ear lobe) so that light is passed through the body part to an optical sensor. The person's oxygen saturation can be estimated based on how the light is absorbed as the light passes through blood flowing in the body part. However, the measurement accuracy of these types of external optical sensor devices can be negatively affected by a person's iron deficiency, their skin pigment, ambient light, among other things.

Certain instances of the present disclosure are accordingly directed to optical sensor devices that can be implanted in a patient.

### Medical Device System

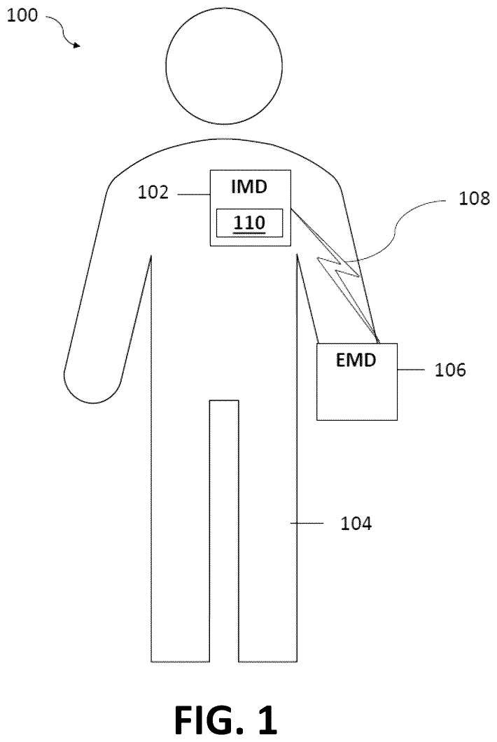

FIG. 1 shows an illustrative medical device system 100, which includes an implantable medical device 102 (hereinafter an “IMD” for brevity) configured to be implanted within the body of a subject 104 and an external receiver or monitoring device 106 (EMD). In the example of FIG. 1, the IMD 102 and the EMD 106 are configured to communicate with one another over a communication link 108.

The IMD 102 may be implanted subcutaneously within an implantation location or pocket in the patient's chest or abdomen and may be configured to monitor (e.g., sense and/or record) one or more physiological parameters. The IMD 102 may be an implantable cardiac monitor (ICM) (e.g., an implantable diagnostic monitor (IDM), an implantable loop recorder (ILR)) configured to record physiological parameters such as, for example, one or more cardiac electrical signals, heart sounds, heart rate, blood pressure measurements, and/or the like. For example, the IMD 102 may include sensors or circuitry for detecting respiratory system signals, cardiac system signals, heart sounds.

The EMD 106 may be a device that is configured to be portable with the subject 104, e.g., by being integrated into a vest, belt, harness, sticker; placed into a pocket, a purse, or a backpack; carried in the subject's hand; and/or the like, or otherwise operatively (and/or physically) coupled to the subject 104. The EMD 106 may be configured to receive data from the IMD 102 and/or monitor physiological parameters associated with the subject 104 and/or provide therapy to the subject 104. In certain instances, the EMD 106 is a cell phone or other device with a user interface that can be used to view aspects of the physiological parameters recorded by the IMD 102.

The IMD 102 includes an optical sensor assembly 110, which can be used to sense one or more physiological parameters, as described in more detail below.

### Implantable Medical Device

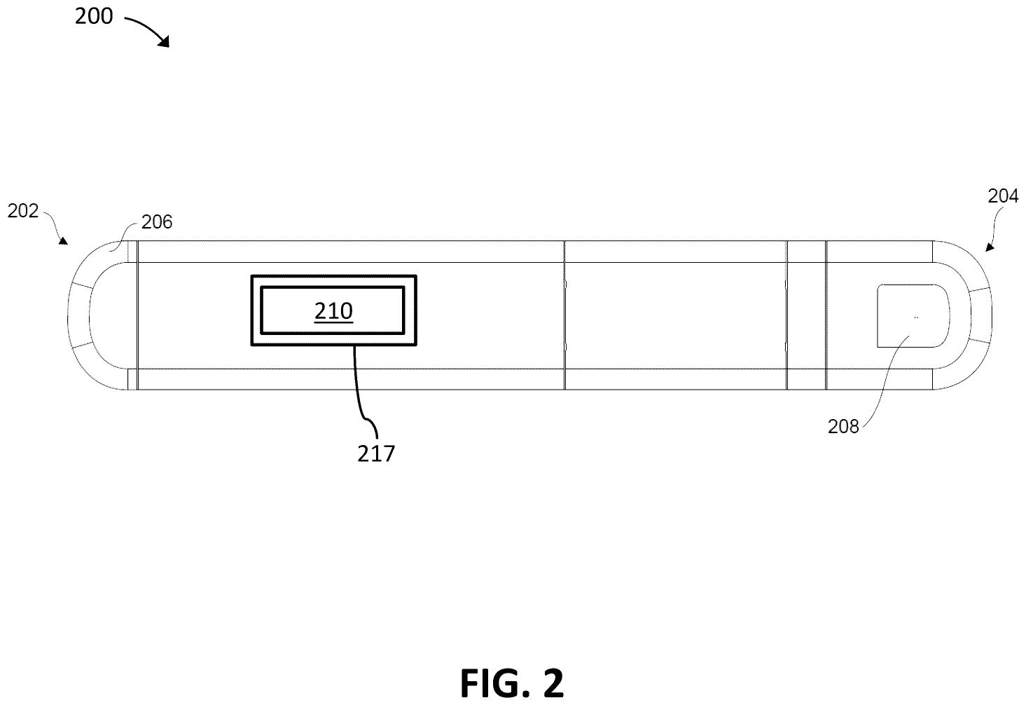

FIG. 2 is a side view of a medical device 200 (hereinafter the “IMD 200”). The IMD 200 may be, or may be similar to, the IMD 102 depicted in FIG. 1 and may be used in the system 100 of FIG. 1.

The IMD 200 includes an external housing that extends between a first end 202 and a second end 204. When assembled, the external housing can create a hermetically sealed enclosure. The IMD 200 can include one or more electrodes 206, 208 for sensing electrical activity of a patient. The IMD 200 can also include features such as an antenna for communicating with external devices, a battery for powering electrical components of the IMD 200, and circuitry, among other features.

FIG. 2 shows the IMD 200 having an optical sensor assembly or optical module 210 (hereinafter the “optical module 210” for brevity). In certain instances, the IMD 200 comprises features described in U.S. patent application Ser. No. 15/242,470 (which is hereby incorporated by reference in its entirety) with the addition of the optical modules described herein.

### Optical Modules

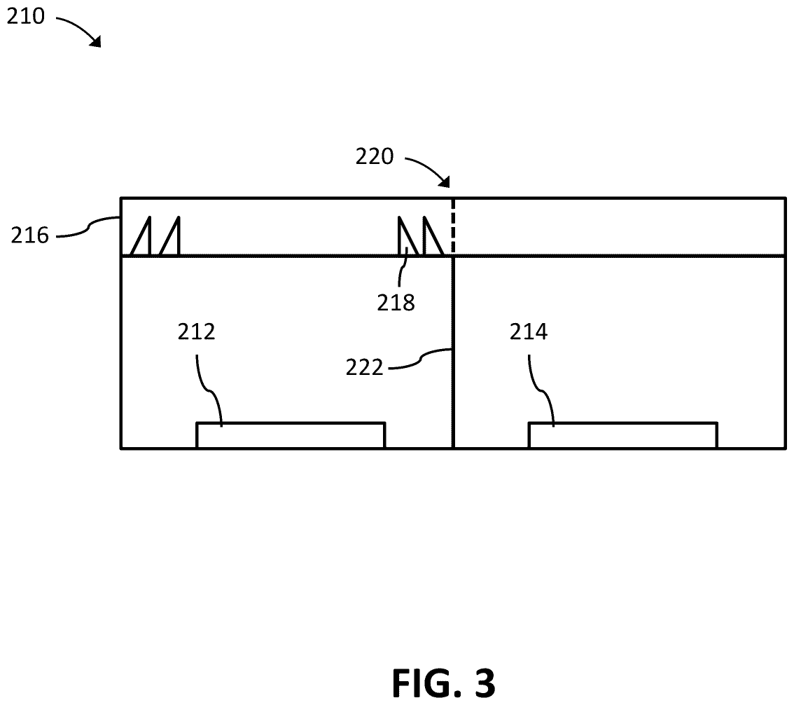

FIG. 3 shows a side cutaway view of one example of the optical module 210. The optical module 210 includes one or more emitters 212 (hereinafter the “emitter 212”) and one or more detectors 214 (hereinafter the “detector 214”). The optical module 210 includes one or more optical layers 216 (hereinafter the “optical layer 216”) that allow light to pass through (e.g., like a window) but that protect the emitter 212 and the detector 214 from the environment external to the optical module 210 or IMD 200. As shown in FIG. 3, both the emitter 212 and the detector 214 have a top surface (e.g., an emitting surface for the emitter 212 and a detecting surface for the detector 214) that faces a bottom surface of the optical layer 216. In certain instances, the optical layer 216 (with its one or more layers) creates a common window for light to both exit and enter the optical module 210—whereas in other instances, the optical layer 216 comprises separate windows (e.g., one or more layers for light exiting and one or more layers for light entering the optical module 210). In either instance, as discussed below, one or more portions of the optical layer 216 can have separate properties or features (e.g., beam steering/filtering/blocking properties or features) than the other portions of the optical layer 216. Instances with a common window may require only one seal 217 (e.g., a seal around the perimeter as shown in FIG. 2), which may be preferable for manufacturability and reliability. Regardless of whether one window or multiple windows are used, the window(s) can comprise glass, sapphire, and the like.

As described in more detail below, the emitter 212 (e.g., one or more light sources such as light-emitting diodes) can be selectively powered such that the emitter 212 emits light out of the optical module 210 and towards a patient's tissue. At least some light will be reflected back (e.g., backscattered light) to the optical module 210 and sensed by the detector 214 (e.g., one or more light sensors such as photodetectors or another type of light sensor). The sensed backscattered light can be used by the IMD 200 to measure physiological parameters such as the patient's blood oxygen content (e.g., SpO2).

There are multiple challenges with emitting and sensing backscattered light with optical sensing devices. One challenge is directing enough backscattered light to the detector 214 due to light being reflected back in directions that are misaligned with the detector 214 such that the reflected light cannot be sensed by the detector 214. To help address this challenge, the optical module 210 can incorporate one or more beam steering features 218.

One example beam steering feature 218 is a lens (e.g., Fresnel lens, contact lens). A lens can be incorporated into an optical layer adjacent to the emitter 212. The lens can direct or focus light in a direction that increases the amount of backscattered light that will ultimately be reflected back to and sensed by the detector 214. Because lenses such as a Fresnel lens can have a rough surface, the lens can be covered by a layer of another optical material to create a smooth outer surface for the optical module 210. Another example beam steering feature 218 is an optical grating designed to direct light in a direction that increases the amount of backscattered light that will be reflected back to and sensed by the detector 214.

Another challenge with optical sensing devices is that non-backscattered light can interfere with the backscattered light. For example, not all light from the emitter 212 may exit the optical module 210 which can cause interference with the backscattered light. To help address this challenge, the optical module 210 can incorporate one or more cross-talk features into the optical layer 216. The example cross-talk features described below are not mutually exclusive and can be used with each other. Similarly, the optical module 210 can use both one or more cross-talk features and one or more beam steering features 218.

As one example of a cross-talk feature, the material and/or dimensions (e.g., thickness) of the optical layer 216 and/or the relative position of the optical layer 216 to the emitter 212 and the detector 214 can be selected to reduce the risk of cross-talk. The relative position of the optical layer 216, the emitter 212, and the detector 214 can be selected such that light is less likely to reflect back towards the emitter 212. Put another way, selection of the relative position of the optical layer 216, the emitter 212, and the detector 214 can help control the amount of light that reflects internally due to its angular relationship with the critical angle of the optical layer 216.

As another example of a cross-talk feature, the optical layer 216 can include a section 220 (represented by a dashed line in FIG. 3) that wholly or partially blocks light that is internally reflected in the optical layer 216. Some emitted light may get “trapped” in the optical layer 216 and get directed towards the portion of the optical layer 216 adjacent to (e.g., directly above) the detector 214. The section 220 can act as a wall or block within the optical layer 216 to help prevent light that is internally reflected within the optical layer 216 from interfering with backscattered light reflected towards the detector 214. For example, the section 220 can include a dark region that is diffused into the optical layer 216. This section 220 of dark material can act like a wall in which the internally reflected light cannot pass through. Additionally or alternatively, the optical module 210 can include one or more walls 222 positioned between the emitter 212 and the detector 214 such that emitted light does not travel within the optical module 210 from the emitter's housing portion to the detector's housing portion.

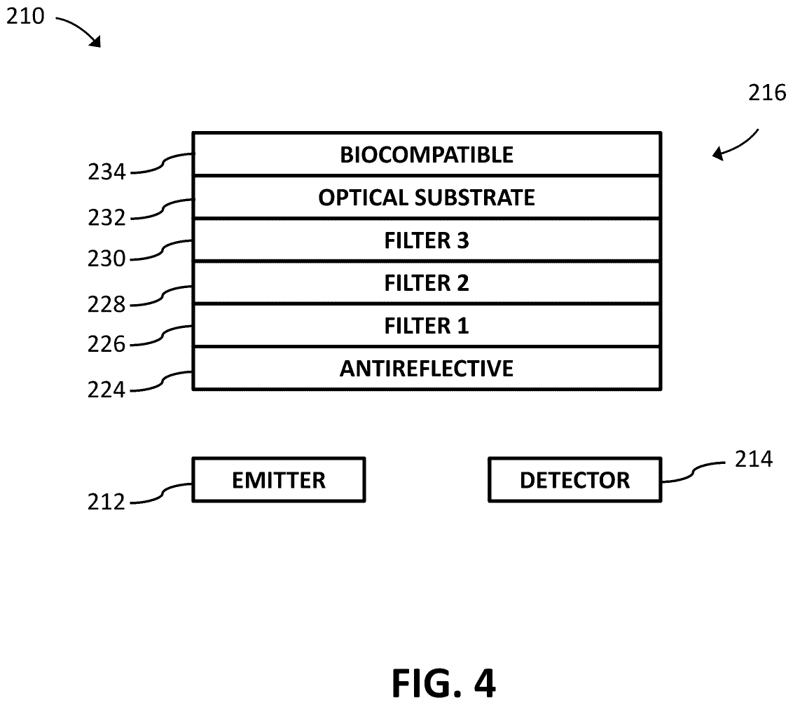

FIG. 4 shows an example layer structure for helping to prevent light from interfering with the backscattered light. As shown in the side view of FIG. 4, the optical layer 216 comprises multiple layers. The number and arrangement of the layers can vary from what is shown in FIG. 4. The layers can include a layer of antireflective material 224 closest to the emitter 212 and the detector 214. One or more optical filter layers 226, 228, and 230 can be positioned between the antireflective layer 224 and an optical substrate 232. The filter layers 226-230 can be designed to help prevent light generated from sources external to the optical module 210 from reaching the detector 214 and/or tune the light generated by the emitter 212. If the IMD 200 is oriented such that the optical module 210 is close to and facing a patient's skin, light such as sunlight can pass through the patient and reach the optical module 210. As such, the filter layers 226-230 can each be designed/selected to filter out certain undesired wavelengths of light (e.g., sunlight, fluorescent light) that are most likely to reach the optical module 210. Additionally or alternatively, one or more optical filters can be coupled directly to the emitter 212 and/or the detector 214. As previously noted, one portion of the optical layer 216 can have different features (e.g., filter(s), doping) than other portions of the optical layer 216. As such, the portion of the optical layer 216 directly above the emitter 212 can use one or more types of filters that are different than the portion of the optical layer 216 directly above the detector 214.

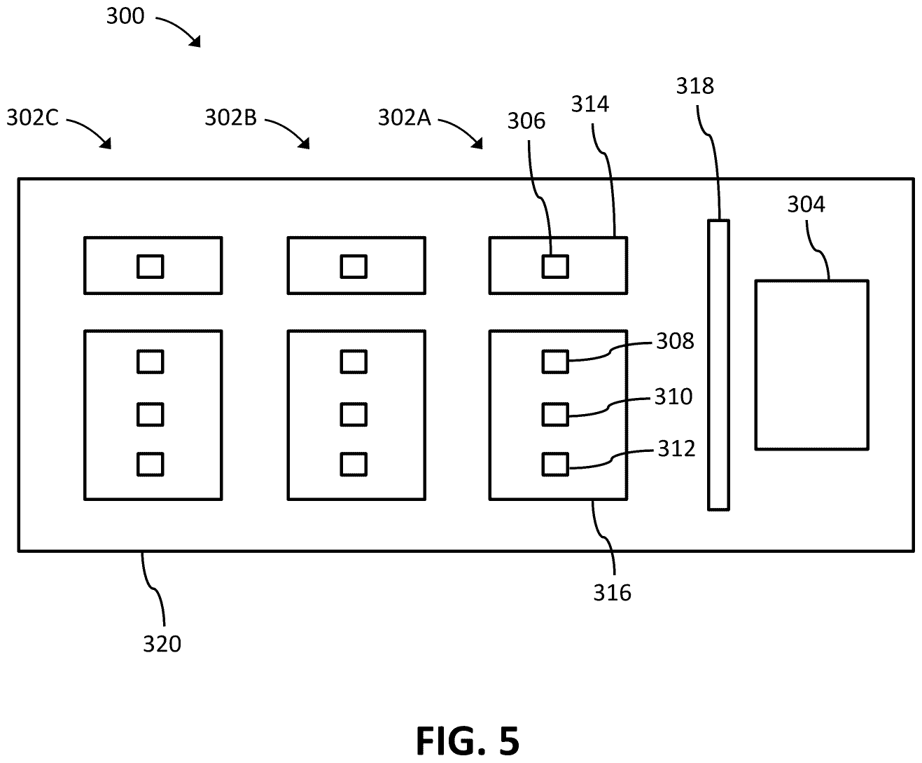

FIG. 5 shows a top view of part of an optical sensor assembly or optical module 300 (hereinafter the “optical module 300” for brevity), which can be incorporated into an IMD such as the IMDs 100 and 200 of FIGS. 1-4. As such, the features shown in FIGS. 2-4 can be used with the features shown in FIG. 5.

The optical module 300 of FIG. 5 includes multiple sets of emitters. For example, the optical module 300 includes a first set 302A of emitters, a second set 302B of emitters, and a third set 302C of emitters. The optical module 300 also includes one or more detectors 304 (e.g., a photodetector or other type of light sensor).

Each set of emitters can include an infrared emitter 306 and one or more visible light emitters such as a blue light emitter 308, a green light emitter 310, and/or a red light emitter 312. Although FIG. 5 shows the infrared emitter 306 as part of one integrated circuit package 314 and the visible light emitters as part of another integrated circuit package 316, the emitters can all be integrated into a single package or separate packages. The optical module 300 also includes a wall 318, which helps prevent light from the emitters from interfering with backscattered light directed towards the detector 304. Each of the components mentioned above can be attached to a substrate 320 such as a printed circuit board or flexible circuit board. When completed, the substrate 320 can be positioned within an IMD or within a separate module that is then incorporated into an IMD. In some instances, the substrate 320 is a single substrate to which all of the above-mentioned components are attached, however, in other instances the components can be attached to separate substrates.

During use, the emitters can be selectively powered to emit light. In some instances, the emitters are selectively powered to determine which emitter or set of emitters provides the best amount (or highest quality) of backscattered light to the detector 304. Because each emitter has a different position relative to the detector 304, each emitter will create a different light path from the respective emitter to the detector 304. The emitters can be cycled through on a 1-by-1 basis or a set-by-set basis or some combination thereof to determine which emitter(s) should be used for measuring and recording physiological parameters. For example, one or more emitters in the first set 302A of emitters can be powered first, followed by the second set 302B, followed by the third set 302C. Circuitry or logic in an IMD, EMD, or another device can determine which set or emitters provides the best amount (or highest quality) of backscattered light to the detector 304 compared to the other sets. For example, the emitter that results in the highest measured amplitude can be ultimately selected for measuring and recording physiological parameters. In instances with multiple detectors 304, different emitter-detector combinations can be tested to see which results in the best amount (or highest quality) of sensed backscattered light.

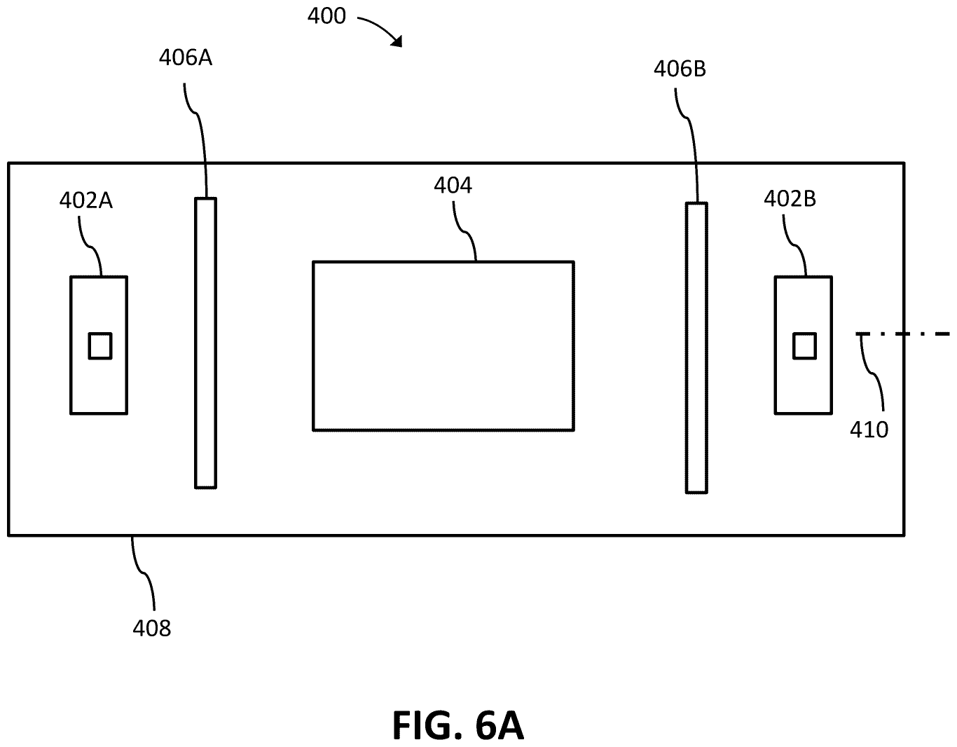

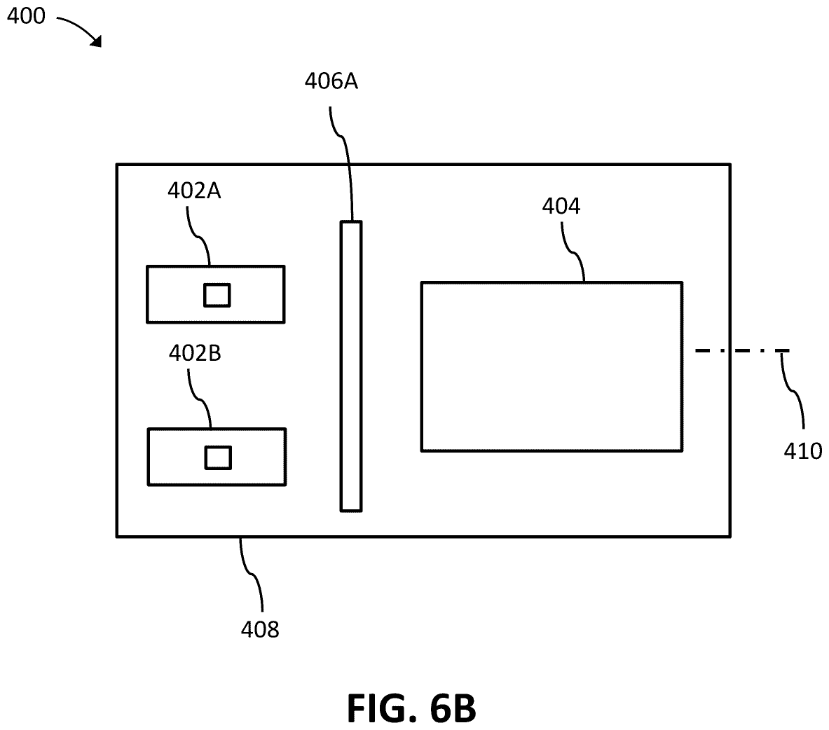

FIGS. 6A and 6B show other example optical modules, which can be incorporated into an IMD such as the IMDs 100 and 200 of FIGS. 1-4. As such, the features shown in FIGS. 1-5 can be used with the features shown in FIGS. 6A and 6B.

Starting with FIG. 6A, an optical module 400 includes a first emitter 402A, a second emitter 402B, one or more detectors 404, a first wall 406A, and a second wall 406B-all of which can be coupled to one or more substrates 408. Although only two emitters and one detector are shown in FIG. 6A, additional emitters and detectors can be used.

In certain embodiments, the first emitter 402A is a light emitting diode (LED) that is designed to emit red light (e.g., wavelengths around 620-750 nm) while the second emitter 402B is an LED that is designed to emit infrared light (e.g., wavelengths around 800 nm to 1 mm). The first emitter 402A and the second emitter 402B can be spaced from the detector 404 along a longitudinal axis 410 of the optical module 400 or an IMD. In the example of FIG. 6A, the detector 404 is positioned between the first emitter 402A and the second emitter 402B, and the respective walls 406A and 406B are each positioned between the detector 404 and one of the emitters 402A and 402B. In some instances, an edge of the first emitter 402A and an edge of the second emitter 402B are positioned 3-6 mm (e.g., 4 mm, 5 mm) from an edge of the detector 404 (e.g., along the longitudinal axis 410 such as a central longitudinal axis of the optical module 400). This range of distances has been found to be sufficient for the detector 404 to receive enough backscattered light from tissue. Although each of the components mentioned above are shown as being coupled to a single substrate 408 (e.g., a single printed circuit board or a single flexible circuit board), the components can be attached to different substrates. Because IMDs are designed to be compact and because the emitters and detector(s) should be spaced from each other, the space between the emitters and detector(s) can be used for other components for the optical module 400 or for the IMD. As such, respective emitters and detector(s) can be coupled to separate substrates so that other components can be more easily positioned between the emitters and detector(s).

FIG. 6B shows the optical module 400 with most of the same components shown in FIG. 6A but rearranged for a more compact design. Because the first emitter 402A and the second emitter 402B are arranged on the same side of the detector 404, the overall length of the optical module 400 can be reduced compared to the design of FIG. 6A. In some instances, an edge of the first emitter 402A and an edge of the second emitter 402B are positioned 3-6 mm (e.g., 4 mm, 5 mm) from an edge of the detector 404 (e.g., along the longitudinal axis 410 such as a central longitudinal axis of the optical module 400).

In any of the examples described above, the emitters can be selected and operated with certain constraints. For example, the emitters can be operated such that surrounding tissue is not heated by the emitted light from the optical module. As another example, the emitters can be collectively powered by no more than 5 mA of current (e.g., at any given point in time) to save power and to help prevent heat generation.

Various modifications and additions can be made to the exemplary instances discussed without departing from the scope of the present invention. For example, while the instances described above refer to particular features, the scope of this invention also includes embodiments having different combinations of features and instances that do not include all of the described features. Accordingly, the scope of the present invention is intended to embrace all such alternatives, modifications, and variations as fall within the scope of the claims, together with all equivalents thereof.

We claim:

1. An implantable medical device (IMD) comprising:
an optical module that includes:
a first light emitter arranged to generate a first beam of emitted light,
a second light emitter arranged to generate a second beam of emitted light,
a light detector arranged to sense backscattered light responsive to the first beam and the second beam, and
at least one optical layer comprising a beam steering feature and/or a cross-talk feature.

2. The IMD of claim 1, wherein the at least one optical layer includes the beam steering feature, wherein the beam steering feature is a lens or an optical grating.

3. The IMD of claim 1, wherein the at least one optical layer includes the beam steering feature, wherein the beam steering feature is a Fresnel lens.

4. The IMD of claim 1, further comprising a first wall positioned between the first light emitter and the light detector, wherein the first wall is arranged to at least partially block non-backscattered light.

5. The IMD of claim 4, wherein the first wall is positioned between the second light emitter and the detector.

6. The IMD of claim 4, further comprising a second wall positioned between the second light emitter and the light detector.

7. The IMD of claim 1, wherein the at least one optical layer includes the cross-talk feature, wherein the cross-talk feature is an area of dark material.

8. The IMD of claim 1, wherein the first emitter and the second emitter are light-emitting diodes.

9. The IMD of claim 1, wherein the at least one optical layer includes the cross-talk feature, wherein the cross-talk feature includes multiple filter layers.

10. The IMD of claim 1, wherein the first light emitter, the second light emitter, and the detector are coupled to a single circuit board.

11. The IMD of claim 1, wherein an edge of the first light emitter is positioned 3-6 mm from an adjacent edge of the detector.

12. The IMD of claim 1, wherein the first beam comprises infrared light, wherein the second beam comprises red light.

13. The IMD of claim 1, further comprising a housing and electrodes, wherein the optical module is positioned within the housing, wherein the electrodes are coupled to the housing.

14. The IMD of claim 1, wherein the at least one optical layer is part of a single window, wherein the single window is arranged such that the first beam, the second beam, and the backscattered light pass through the single window.

15. The IMD of claim 14, further comprising a single seal surrounding a perimeter of the single window.

16. The IMD of claim 1, wherein the optical module is configured to consume a maximum of 5 milliamps of power during operation.

17. The IMD of claim 1, wherein the light detector is a single light detector, wherein the light detector is the only light detector of the IMD.

18. An implantable medical device (IMD) comprising:
an optical module that includes:
a first light emitter arranged to generate a first beam of emitted light,
a second light emitter arranged to generate a second beam of emitted light,
a light detector arranged to sense backscattered light responsive to the first beam and the second beam, and
means for steering the first beam and the second beam.

19. The IMD of claim 18, further comprising means for reducing interference with the backscattered light.

20. The IMD of claim 19, wherein the first beam comprises infrared light, wherein the second beam comprises red light.
